(19) United States
(12) Patent Application Publication
(10) Pub. No.: US 2026/0144826 A1
(43) Pub. Date: May 28, 2026

(54) METHOD FOR LOW-DENSITY EXPANSION OF HUMAN CARTILAGE CELLS

(71) Applicant: OSPEDALE GALEAZZI S.P.A., MILANO (IT)

(72) Inventors: Alessandra COLOMBINI, MILANO (IT); Silvia LOPA, MILANO (IT); Francesca LIBONATI, MILANO (IT); Laura DE GIROLAMO, MILANO (IT); Matteo Giovanni MORETTI, MILANO (IT)

(21) Appl. No.: 18/996,445

(22) PCT Filed: Jul. 24, 2023
(86) PCT No.: PCT/EP2023/070435

§ 371 Date: Jan. 17, 2025

(30) Foreign Application Priority Data
Jul. 25, 2022 (IT) 102022000015615

(51) Int. Cl. A61K 35/32; A61P 19/02; C12N 5/077

(52) U.S. Cl. CPC A61K 35/32; A61P 19/02; C12N 5/0655; C12N 2500/84

(57) ABSTRACT

It is an object of the present invention a method to obtain a cartilage cell population enriched in cartilage progenitor cells (CPCs), said method comprising culturing said cells in culture at a density between 10 and 500 cells/cm². In a further aspect, it is here claimed a population of cartilage cells enriched in CPCs obtainable by means of said method, and their use in the treatment of chondral lesions or joint degeneration.

This application is the U.S. national phase of International Application No. PCT/EP2023/070435 filed Jul. 24, 2023 which designated the U.S. and claims priority to IT Patent Application No. 102022000015615 filed Jul. 25, 2022, the entire contents of each of which are hereby incorporated by reference.

In a comprehensive and standardized clinical classification, osteoarthritis (OA) is defined as “a disorder involving movable joints characterized by cell stress and extracellular matrix degradation, initiated by micro- and macro-injury that activates maladaptive repair responses including pro-inflammatory pathways of innate immunity” (Kraus V B, et al. Osteoarthritis and cartilage. 2015; 23(8):1233-41).

OA is the most common cause of joint degeneration, with a worldwide prevalence of 10%, increasing up to 33% in the elderly, and, given the aging population, it represents an increasing social burden (Cross M, et al. Annals of the rheumatic diseases. 2014; 73(7):1323-30). Joint degeneration is also a hallmark of a rare disease, the camptodactyly-arthropathy-coxa vara-pericarditis syndrome (CACP). CACP is an autosomal recessive condition of children caused by homozygous mutation in the proteoglycan-4 gene (PRG4) characterized by the association of congenital or early-onset camptodactyly and non-inflammatory arthropathy.

The currently available symptomatic treatments for OA, such as physical therapy, hyaluronic acid injection and anti-inflammatory drugs, suffer of poor long-term efficacy and cannot reverse the progressive joint degeneration, leading clinicians to joint replacement surgery (Katz J N, et al. N Engl J Med. 2013; 368(18):1675-84).

Joint replacement is a highly invasive procedure, determining limited articular mobility, which should be therefore avoided, especially in young patients with an active lifestyle.

An effective treatment of OA in its early phases could avoid the pathology progression and allow postponing the need for more invasive interventions. In this clinical scenario, the development of alternative, conservative approaches based on cell therapy is particularly relevant.

The first widely accepted cell-based regenerative treatment for cartilage repair, used for over 20 years, is the autologous chondrocyte implantation (ACI) (Brittberg M, et al. N Engl J Med. 1994; 331(14):889-95; O'Sullivan J, et al. Stem cell research & therapy. 2011; 2(1):8). This approach has been safely used in clinical practice for the treatment of focal traumatic chondral lesions and has been proposed also as a suitable therapeutic option for the treatment of patients with early OA (Colombini A, et al. Knee Surg Sports Traumatol Arthrosc. 2023; 31(6):2338-2348).

Despite being a valid approach, ACI has different limitations that should be mitigated. ACI implies two in vivo steps: a first surgery for the collection of cartilage pieces and a second surgery for the treatment of the cartilage defect(s) with the chondrocytes that in the interval between surgeries have been isolated from cartilage and expanded in a good manufacturing practice (GMP) laboratory.

In this procedure, chondrocytes are isolated from a healthy cartilage area. This is more relevant and represents one of the major limiting factors of this technique when the defect area to treat is wide, as this would require harvesting a large cartilage fragment. In fact, the number of cells that can be generated depends on the dimension of the cartilage fragment (Khan I M, et al. Osteoarthritis and cartilage. 2009; 17(4):518-28).

Given the scarce cellularity of cartilage, extensive in vitro cell expansion is hence needed to achieve a sufficient number of chondrocytes to perform the technique. During this step, chondrocytes tend to lose their chondrogenic phenotype (Khan I M, cit; Williams R, et al. PLoS One. 2010; 5(10):e13246; Coumil-Henrionnet C, et al. Biorheology. 2008; 45(3-4):513-26), leading to the formation of a repair tissue that is often fibrocartilage, that is biomechanically inferior to native articular cartilage, thus jeopardizing the long-term repair of the cartilage defect (Peterson L, et al. Clin Orthop Relat Res. 2000; 374:212-34; Tuli R, et al. Arthritis Res Ther. 2003; 5(5):235-8; Roberts S, et al. Arthritis Res Ther. 2003; 5(1):R60-73).

Beyond mature chondrocytes, which were long considered the only cell type within articular cartilage, a population of cartilage cells with migratory, clonogenic ability and differentiation potential, termed as chondroprogenitor cells (CPCs), has been also found both in healthy and damaged cartilage (Jiang Y, Tuan R S. Nat Rev Rheumatol. 2015; 11(4):206-12; Koelling S, et al. Cell Stem Cell. 2009; 4(4):324-35). In view of the ‘stem-like’ or ‘progenitor-like’ properties of CPCs, it has been proposed that these cells might have a role in cartilage repair.

Cartilage cells derived from cartilage of OA patients and containing CPCs have revealed a noteworthy chondrogenic potential, active secretory response, and strong immunomodulatory function after inflammatory priming. Remarkably, with increasing passages in culture, these cells are characterized by a progressive enhancement of clonogenic ability and sustained expression of stemness markers (De Luca P, et al. J Clin Med. 2019; 8(7):975). These features indicate an enrichment in CPCs throughout the in vitro expansion of the whole cell population.

Although CPCs express several mesenchymal surface markers, such as CD105, CD166 and CD146, these markers are expressed also by mature chondrocytes, and consequently cannot be used to selectively isolate CPCs. Hence, there is an unmet need for a robust protocol to enrich cartilage cell cultures in CPCs, exploitable for the improvement of the existing cartilage repair procedures or for the development of new cell-based therapies for the treatment of both chondral lesions and early osteoarthritis.

Moreover, cell manufacturing processes intended for clinical application on humans must comply with European cell therapies regulations, avoiding the usage of animal sera and replacing them with human alternatives (Sykes J G, et al. Eur Cell Mater. 2018; 35:255-267). Indeed, although fetal bovine serum (FBS) is widely used for human cell cultures (Hemeda H, et al. Cytotherapy. 2014; 16(2):170-80), its clinical application has raised safety concerns, such as the development of immune responses after administration of cells expanded in xenogenic sera (Spees J L, et al. Mol Ther. 2004; 9(5): 747-56) or against fungi, bacteria, viruses, or prions (Jochems C E A, et al. Altern Lab Anim. 2002; 30(2):219-27). In ACI procedure human autologous serum is currently used as supplement during in vitro cell expansion. As an alternative, human platelet lysate (hPL) can be used as human supplement standardized in growth factor concentration and characterized by low batch-to-batch variability, being developed from multiple donors (Castiglia S, et al. Cytotherapy. 2014; 16(6):750-63; Mohamed H E, et al. Blood Res. 2020; 55(1):35-43).

### DESCRIPTION

It is an object of the present invention a low-density culture method, which exploits cell clonogenic potential, to obtain a population of cartilage cells enriched in CPCs.

In an embodiment, this innovative culture method is combined with the use of allogeneic human platelet lysate (hPL) as adjuvant for cell expansion to promote cell growth and potential. A major advantage of hPL is that it allows avoiding a consistent blood sampling from the patients to obtain autologous serum.

This method, based on low density culture, allows obtaining a high number of cells, suitable for the treatment of chondral defects of large dimensions or diffuse joint degeneration processes, even starting from a very low cell number (corresponding to a small portion of cartilage to harvest). Additionally, this method allows selecting cells with superior chondrogenic potential compared to cells cultured at standard density.

In an embodiment, a population of cartilage cells enriched in CPCs is claimed.

### DRAWINGS DESCRIPTION

FIG. 1: Senescence analysis. Representative images of R-galactosidase activity. Cells cultured according to the invention, at low density in hPL (L); cells cultured at standard density in hPL (comparative) (C).

FIG. 2: Karyotype of cells cultured according to the invention, at low density in hPL, after 42 days of culture; chromosomes are arranged in numerical order.

FIG. 3: Surface marker expression. (A-E) FACS analysis for the indicated markers, performed on cells from 5 Donors (01-05) cultured according to the present invention (L) or at standard density in hPL (comparative) (C). Black curve: unstained cells. Grey curve: stained cells.

FIG. 4: Representative images of cells cultured according to the present invention (L) or at standard density in hPL (C) and stained with haematoxylin and eosin (HE), Alcian Blue (glycosaminoglycan staining) and for type I and II collagen.

FIG. 5: Gene array of a panel of markers on cells from 4 Donors (01-02-04-05). Data are reported in a heat map showing the fold change (Fc) of the gene expression of cells cultured according to the present invention (L) in comparison with standard density cultured cells in hPL (C). Histograms show the significant differences between the expression of CD146, GREM1 and TIMP2 in the two population of cells. Data are showed as dCt of cells cultured according to the present invention (L) or of standard density cultured cells in hPL (C).

FIG. 6: Surface marker expression. (A-D) FACS analysis for CD166 and CD146 markers performed on cells from 4 Donors (06-09) cultured according to the present invention (L) or at standard density in hPL (comparative) (C) for 14 days. Black curve: unstained cells. Grey curve: stained cells.

FIG. 7: Macrophage surface marker expression. FACS analysis for CCR7, CD80, CD206 and CD163 performed on macrophages co-cultured with cells from 5 Donors (01-05) cultured according to the present invention (L) or at standard density in hPL (comparative) (C).

### DETAILED DESCRIPTION

In a first embodiment, it is here claimed a method to obtain a cartilage cell population enriched in CPCs, said method comprising:
Collecting cartilage fragments;
Isolating cartilage cells;
Plating said cartilage cells at a standard density in culture medium;
Culturing said cartilage cells;
Once confluence is reached, collecting the cells, and plating them in culture medium at low density, between 10 and 500 cells/cm², and culturing until use.

When referring to “standard density”, a cell density in a range comprised between 3.000 and 50.000 cells/cm², or between 5.000-30.000 cells/cm², or 6.000 and 10.000 cells/cm² is intended.

In a preferred embodiment, when referring to “low density”, a cell density in a range comprised between 20 and 100 cells/cm², or around 50 cells/cm² is intended.

In an embodiment, said culture medium comprises blood derivatives, selected from the group comprising platelet concentrates, platelet lysates (PLs), and serum. In an embodiment, said blood derivatives originate from mammals. In an embodiment, said blood derivatives originates from human. In an embodiment, said serum is foetal serum, in an embodiment, said serum is autologous serum.

In a preferred embodiment, said blood derivative is hPL.

In an embodiment, said hPL is obtained from blood banks, cell factories or research laboratories. In an embodiment, said hPL is commercially available.

In an embodiment, said hPL is used immediately after preparation (fresh). In an embodiment, said hPL is used after cryopreservation. In an embodiment, said hPL freeze-dried is used after reconstitution.

In an embodiment, said blood derivatives are added to the culture medium at 2-20% (V/V), or at 5-10%.

In an embodiment, said cartilage cell culture is performed in standard condition: 37° C., 5% CO₂, relative humidity 95%.

To expand the cellular population, once the cells cultured at low density reach the confluence, they are detached and re-plated at low density.

In an embodiment, said tissue is obtained from a donor without joint diseases. In an embodiment, said tissue is obtained from donors with joint diseases, such as OA.

In an embodiment, cartilage cells are isolated by mechanical disruption. In an embodiment, cartilage cells are isolated by enzymatic digestion. In an embodiment, cartilage cells are isolated by a combination of these two methods (mechanical disruption and enzymatic digestion).

Optionally, once isolated said cartilage cells are frozen until use.

In a second embodiment, it is here claimed a population of cells obtained according to the here described method.

The Authors of the present invention surprisingly observed that starting from the same number of cells seeded at day 0, a 10-fold higher cell number was obtained at day 10 using a culture method according to the invention (L), compared to culture at standard density (C). In line with this result, and even more remarkably, a 100-fold higher cell number was obtained when culturing the cartilage cells according to (L) in comparison with (C) at day 20.

Cells obtained according to the method of the invention show a stable karyotype. Moreover, their immunogenicity, intended as the ability of cells to provoke an immune host response upon implantation, is low, since their immunological profile resembles the one of mesenchymal stromal cells. Immunohistochemical analysis revealed a higher presence of type I and II collagen, typical markers of articular cartilage matrix, in the cells obtained according to the invention and chondrogenically differentiated with respect to those cultured according to state-of-the-art methods.

Cells obtained by the method according to the present invention have been characterised by gene expression and flow cytometry analysis. Surprisingly, the cells are characterised by a distinctive pattern of gene and protein expression with respect to their counterpart obtained according to the state of the art methods.

It forms a further object of the present invention a cartilage cell population enriched in CPCs, wherein said cells express higher levels of the mesenchymal surface marker CD146, both at gene and protein level, with respect to cartilage cells obtained according to the state-of-the-art methods.

In an embodiment, the cell population according to the present invention is characterized by the following expression profile, with respect to cells obtained according to the state-of-the-art methods:
Increased expression: COMP, CD146, DKK1, CD166;
Decreased expression: MMP13.

In an embodiment, said expression profile comprises:
Increased expression: COMP, CD146, DKK1, CD166, MMP3, GREM1, TGFBR2;
Decreased expression: TIMP2, PRG4, BMPR1B, MMP13.

If forms a further object of the present invention spheroids comprising cells according to the present description. As an example, said spheroids are obtained by seeding cells on non-adhesive supports or by collecting them into spheroids by centrifugation.

The here described method, and the cells obtained, are advantageous tools for improving ACI and for developing allogeneic procedures for the treatment of chondral lesions.

In an embodiment, the here described cells are claimed for use in the treatment of chondral lesions or articular degeneration.

Moreover, it is here claimed the allogenic use of the here described cells to treat the joints of the CACP patients. Beyond regenerating the cartilage tissue, the cells of the present invention represent a source of PRG4, able to lubricate the joint, which is missing in CACP patients.

The method according to the invention surprisingly avoids an extensive expansion of cartilage cells, responsible of phenotype loss, while still obtaining a sufficient number of cells to treat chondral defects of large dimensions or diffuse joint degeneration.

Moreover, the method according to the present invention complies with one of the major requirements of European cell therapies regulations. In fact, in an embodiment, the instant claimed method does not use animal sera, wherein the same is replaced with human alternatives. Specifically, the here proposed method implies the use of hPL, a safe cell culture supplement characterized by consistent growth factor concentration and low batch-to-batch variability. Additionally, the use of allogeneic hPL allows overcoming the limitations related to the use of human autologous serum related to the withdrawal of large amount of blood from the patient to obtain autologous serum.

Remarkably, the possibility to select CPCs also from cartilage tissue collected as surgical waste material from OA donors, as a cheaper and more convenient cell reservoir, allows an allogeneic use of these cells.

The following examples do not limit the scope of the protection of the present invention, whose scope of protection is defined by the attached claims.

### Materials and Methods

Preparation of Human Platelet Lysate (hPL)

The platelet pool of 60 healthy donors was used to prepare hPL bags. The donors were tested for ABO blood groups, irregular red blood cell antibodies and infectious markers (hepatitis B and C, human immunodeficiency virus 1/2 and Treponema pallidum).

Briefly, in a triple-bag system (Fresenius Kabi, Bad Homburg, Germany) containing citrate-phosphate-dextrose, 450±45 mL of whole blood was collected. The blood units were separated by centrifugation with an automated separator (Compomat G5, Fresenius Kabi) to obtain the blood components: plasma, buffy coat and red blood cells. 4 O-group buffy coats with AB-group plasma were pooled to obtain the buffy coat-platelet concentrate (BC-PC), followed by leukocyte depletion through centrifugation and filtering by TACSI system (Terumo BCT Europe, N.V. Zaventem, Belgium) and pathogens inactivation through Mirasol PRT System (Terumo BCT Europe). A platelet concentration of 1026×10⁶/μl was determined by Sysmex XE-2100, while platelet fragmentation and growth factor release were obtained by three cycles of freezing (−35° C.) and thawing (37° C.) of BC-PC. Finally, BC-PC was centrifugated (5000 g, 8 min), platelet bodies were removed, and the supernatant was collected.

Each batch of hPL was then divided into aliquots and frozen at −20° C. until use.

### Isolation, Cryopreservation and Low-Density Expansion of Cartilage Cells

The study involved, with informed consent, 5 patients (3 male and 2 female, age range: 41-55 years, mean age: 47 years) who underwent arthroplasty.

The articular cartilage (AC) was cut into small pieces under sterile condition and enzymatically digested with 0.15% (w/v) type II Collagenase (Worthington Biochemical Corporation, Lakewood, NJ, USA), for 22 hours at 37° C. under stirring. The digested tissue was filtered with cell strainer (100 μm mesh) to remove tissue residuals and collect human cartilage cells. After isolation, cartilage cells were counted with Burker chamber diluting the suspension 1:1 with red blood cell lysis solution (Trypan Blue+3% acetic acid) and cryopreserved at −80° C. in cryoprotective solution consisting of hPL with 10% DMSO (Sigma-Aldrich, St. Louis, Missouri, USA).

After thawing, cells were seeded at a density of 10000 cells/cm² in expansion medium made of High Glucose (HG) Dulbecco's modified Eagle's medium (DMEM) (Sigma-Aldrich) supplemented with 10% (V/V) hPL, 10 mM HEPES, 1 mM sodium pyruvate, 200 mM L-Glutamine, 100 U/mL penicillin, 100 μg/mL streptomycin (all reagents from Thermofisher Scientific, Waltham, MA, USA) and incubated at 37° C., 5% CO₂. After 7 days, when confluence was reached, the cells were split and expanded at normal (C) (5000 cells/cm²) and low density (L) (50 cells/cm²) in expansion medium, with standardized detachment time, 7 days for normal-density cultured cells and 14 days for low-density cultured cells. The cells were expanded up to three passages (indicated as P3) and used for the subsequent experiments.

### Senescence Assay

A senescence assay based on the R-galactosidase activity (SA-βGal) was performed after 42 days of culture at both low and standard density to exclude cellular senescence potentially induced by the in vitro expansion of cartilage cells. SA-βGal activity was measured by a senescence detection kit (BioVision, Milpitas, CA, USA), according to the manufacturer's instruction. Briefly, cells were fixed for 10 min at room temperature and stained with a solution containing X-gal at 37° C. overnight. The next day, cells were visualized using an inverted Olympus IX71 microscope.

### Karyotype Analysis

To exclude cytogenetic transformation during in vitro expansion, cartilage cells were analysed after 42 days of culture at low density in hPL, according to a standard method. Briefly, the cells were arrested at the metaphase by incubation with Colcemid (Invitrogen Corporation, Grand Island, NY, USA) and then maintained in a hypotonic solution (0.075 mol/L KCl), fixed with methanol/acetic acid 3:1 (Merck, Milan; Italy) and stained with Giemsa. Cells in metaphase were analyzed with the use of MackType software (Nikon Corporation, Tokyo, Japan) according to the International System for Human Cytogenetic Nomenclature.

### Expression of Surface Markers

A flow cytometry analysis was conducted at P3 on 3×10⁵ cartilage cells, cultured at low or standard density. In particular, the expression of CD166, a marker which was found to correlate with the clinical success of ACI (Islam A. et al. BMC Musculoskeletal Disorders 2019, 20, 19), CD146, that denotes a perivascular phenotype (Crisam M et al. Cell Stem Cell 2008, 3(3):301-13) and MHC I and II, markers of immunogenicity was analysed. The cells were washed and resuspended several times with cold FACS Buffer (13.7 mM NaCl, 0.27 Mm KCl, 0.43 mM Na₂HPO4, 0.14 mM KH₂PO4, 2% FBS, 0.1% NaN₃ in ddH₂O, pH: 7.4) and then incubated for 30 minutes with CD166 conjugated FITC (Ancell Corporation, Bayport, MN, USA), CD146 conjugated APC/Fire, HLA-A, B, C (MHC-I) conjugated APC and HLA-DR (MHC-II) conjugated PER-CP (BioLegend, San Diego, CA, USA) anti-human monoclonal antibodies diluted 1:50 in 100 μl of FACS Buffer. Background fluorescence was established by unstained cells as negative controls and data were acquired using a FACS Calibur flow cytometer (BD Biosciences, San Jose, CA, USA) collecting a minimum of 10.000 events.

To confirm the data concerning the higher gene expression of CD146 and CD166, a flow cytometry analysis was conducted on 3×10⁵ cartilage cells cultured at low or standard density for 14 days.

### Culture in Pellet

After three passages in culture, 4×10⁵ cartilage cells expanded at standard and low density were centrifuged (5 minutes at 600 g) and maintained for 14 days in expansion medium or in chondrogenic medium consisting of serum-free expansion medium supplemented with 2.50 μg/mL amphotericin B, 1.25 mg/mL human serum albumin, 10 μg/mL insulin from bovine pancreas, 5.5 μg/mL human transferrin, 5 ng/mL sodium selenite, 0.5 mg/mL bovine serum albumin, and 4.70 μg/mL linoleic acid, 0.1 μM dexamethasone, 0.1 mM L-ascorbic acid-2-phosphate (all from Sigma-Aldrich), and 10 ng/mL TGF beta 1 (Peprotech, Rocky Hill, NJ, USA), namely chondrogenic medium.

### Histological Analysis

Pellets were fixed with 10% neutral buffered formalin (Sigma-Aldrich, St. Louis, Missouri, USA), rinsed in phosphate-buffered saline (PBS), placed in 70% ethanol, then embedded in paraffin and sectioned at 4 μm thickness. Sections were stained using haematoxylin and eosin (Carlo Erba, Milan, MI, ITA) to evaluate matrix deposition and cell morphology, or alcian blue pH 2.5 (Sigma-Aldrich) to evaluate the glycosaminoglycan content. The pellet sections were also assessed for the expression of Type I and II collagen. Briefly, sections were blocked with 2% bovine serum albumin (BSA) (Sigma-Aldrich), and incubated with a rabbit monoclonal anti-collagen type 1, 1:4000 (ab138492, Abcam, Cambridge, CB4 0FL, UK), rabbit polyclonal anti-collagen type II, 1:100 (ab34712, Abcam) diluted in 5% BSA in PBS, for 1 hour at room temperature. After incubation, the sections were washed with PBS added with Tween20 and incubated for 30 minutes with 1:200 anti-rabbit IgG, (H+L) raised in goat, biotinylated secondary antibody diluted in PBS 2% BSA, (VC-BA-1000-MM15, Vector Laboratories, Burlingame, CA, USA). Diaminobenzidine (Vector Labs, Burlingame, CA, USA) was used as a chromogenic substrate of the peroxidase reaction. The Bern Score visual grading system was used to assess the chondrogenic differentiation (Grogan S P et al. Tissue Engineering 2006, 12, 8) and immunostained sections were scored for the presence of type I and II collagen by using a semi-quantitative scoring system (Colombini A et al. Int. J. Biochem. Cell Biol. 2012, 44:1019-1030) as follows: 0=absence, 1=mild, 2=moderate, 3=marked.

### Gene Array Analysis

After expansion for 14 days at normal- and at low-density culture, cells were collected for RNA extraction. RNA was extracted RNeasy Mini Kit (Qiagen, Duesseldorf, Germany). For residual genomic DNA digestion, RNase-Free DNase Set (Qiagen) was used and the isolated RNA was quantified spectrophotometrically (Nanodrop, Thermo Scientific, Rockford, IL, USA). A panel of 31 selected genes of interest was investigated trough Taqman gene expression array (Thermofisher Scientific, Waltham, MA, USA) following the manufacturer's protocol by using QuantStudio Real-Time PCR System. TBP and YWHAZ were used as housekeeping genes for data normalization.

### Evaluation of the Immunomodulatory Potential of the Cartilage Cells

After thawing, monocytes obtained from 10 donors were pooled together. Cells were seeded at a density of 2×10⁵ cells/cm² in RPMI 1640 (Sigma-Aldrich) added with 100 U/mL penicillin, 100 μg/mL streptomycin, 200 mM glutamine (ThermoFisher Scientific) and 10% (v/v) hPL.

To differentiate monocytes into macrophages, 20 ng/ml of macrophage colony-stimulating factor (M-CSF, Peprotech Inc, Rocky Hill, NJ, USA) were added to hPL-supplemented medium for 5 days. At day 5, the chondrocytes expanded at normal- and at low-density were thawed, seeded in transwell (1.5×10⁴ cells/cm²) and cultured in chondrocyte culture medium. After 2 days (day 7), the transwell were transferred to the macrophage-seeded 6-well plates to initiate the co-culture. Chondrocytes and macrophages were co-cultured for 2 days using a mix of macrophage and chondrocyte medium (1:1). At the end of the co-culture, macrophages were detached, stained for cell surface markers and analyzed by flow cytometry. Monocytes differentiated for 7 days into M0 macrophages and treated with 10⁻⁷ M dexamethasone (Sigma-Aldrich) were used as anti-inflammatory control to validate the model.

The expression of M1 (CD80 and CCR7) pro-inflammatory, M2a (CD206) and M2c (CD163) anti-inflammatory markers was evaluated at day 9 by flow cytometry analysis. Briefly, macrophages were washed twice with PBS, detached by incubation with cell dissociation buffer (Thermo Fisher) for 7 min and centrifuged at 500 g for 5 min. Cells were then suspended in MACS buffer (Miltenyi Biotec), treated with FcR Blocking Reagent (Miltenyi Biotec) for 10 min at 4° C. to block unwanted binding of antibodies to human Fc receptor and counted. Antibodies anti-human CD80-APC (Clone REA661, Miltenyi Biotec) and CCR7-PE-Vio770 (Clone REA108, Miltenyi Biotec) for M1 phenotype, anti-human CD206-FITC (Clone 15-2, Biolegend) for M2a phenotype, and anti-human CD163-PE (Clone GHI/61, Biolegend) for M2c phenotype were used. Unstained cells were used as negative control for fluorescence. All the stains were performed at 4° C. for 20 min in the dark. Data were acquired using a Cytoflex flow cytometer (Beckman Coulter, Brea, CA, USA). Cells were stained with 1 μg/mL DAPI for 5 min in the dark at room temperature to assess viability.

### EXPERIMENTAL RESULTS

### Example 1: Cell Proliferation and Senescence Analysis

Table 1 reports the data related to cell proliferation. Cells obtained from 5 donors were cultured according to the invention (first block, L), at standard density in hPL (comparative, second block, C), or at standard density in FBS (comparative, third block, FBS C).

Cells cultured according to the present invention (L) form colonies and exhibit a number of population doubling/day that is 1.7- and 4-fold higher than cells cultured at standard density in hPL (C) or cultured at standard density in FBS (FBS C), respectively. The superior proliferative ability of cells cultured according to the present invention (L) corresponds to an inferior number of hours required for each population doubling compared to C and FBS C (h/Po. Doublings).

TABLE 1

| | | | |
|---|---|---|---|
| | | Pop. | h/Pop. |
| Donor | Donor | Doublings/Day | Doublings |
| sex | Age | P1 | P1 |

L

| | | | | |
|---|---|---|---|---|
| D05 | F | 55 | 0.89 | 26.9 |
| D06 | M | 46 | 0.60 | 40.0 |
| D14 | M | 41 | 0.59 | 40.5 |
| D16 | F | 49 | 0.58 | 41.1 |
| D17 | M | 44 | 0.90 | 26.6 |
| Mean | | | 0.71 | 35.0 |
| SD | | | 0.17 | 7.6 |

C

| | | |
|---|---|---|
| D05 | 0.38 | 62.6 |
| D06 | 0.56 | 42.9 |
| D14 | 0.42 | 56.7 |
| D16 | 0.47 | 51.4 |
| D17 | 0.31 | 77.5 |
| Mean | 0.43 | 58.2 |
| SD | 0.09 | 13.0 |

FBSC

| | | |
|---|---|---|
| C9 | 0.14 | 169.2 |
| C4 | 0.20 | 120.8 |
| C10 | 0.20 | 121.1 |
| C6 | 0.15 | 155.3 |
| C5 | 0.19 | 127.4 |
| Mean | 0.18 | 138.7 |
| SD | 0.03 | 22.2 |
| t test L vs C | * | ** |
| t test L vs FBS C | ** | *** |

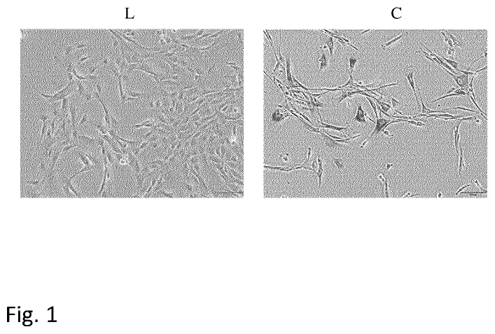

The evaluation of cell senescence was performed on the same cells from the 5 donors cultured for 42 days. Representative images of β-galactosidase expression are reported in FIG. 1. The staining is clearly increased in control conditions (C), indicating the presence of more senescent cells. Cells cultured according to the present invention (L) appear healthier.

### Example 2: Karyotype Stability

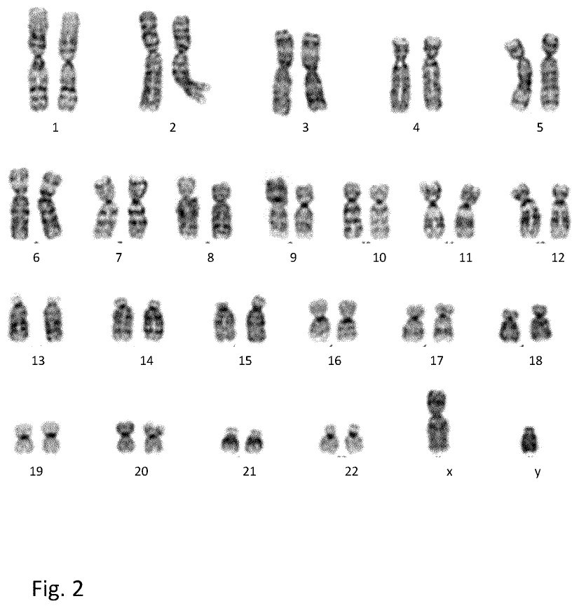

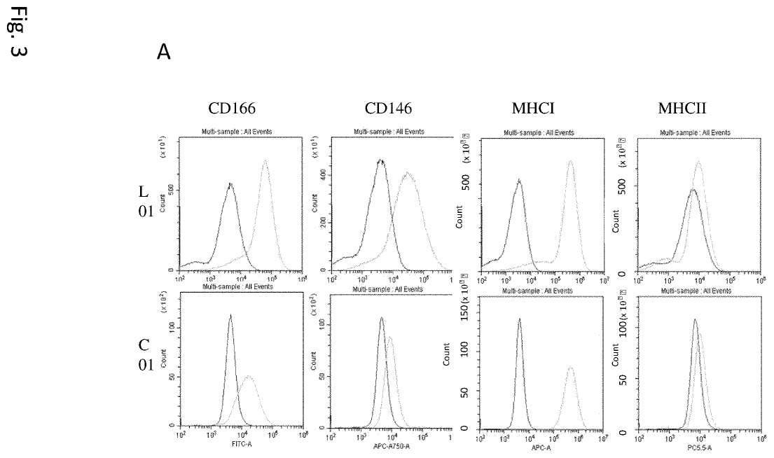

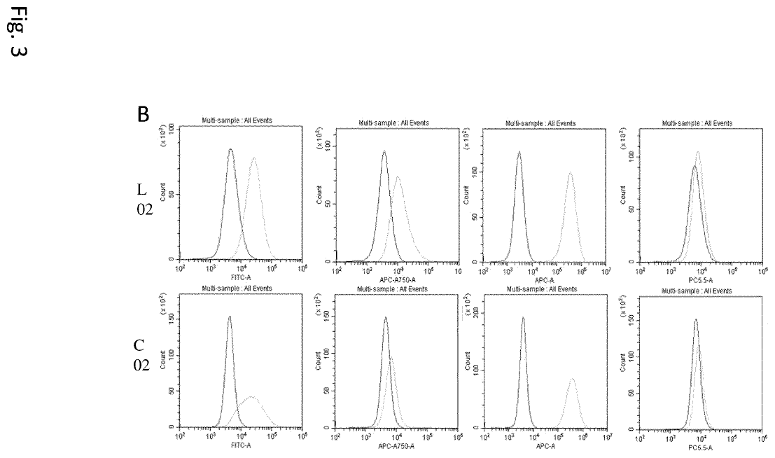

The cells cultured according to the present invention show karyotype stability after 42 days of culture (FIG. 2).

### Example 3: Surface Marker Expression

Cells cultured according to the invention (L) or according to the control condition (C) were evaluated for surface marker expression.

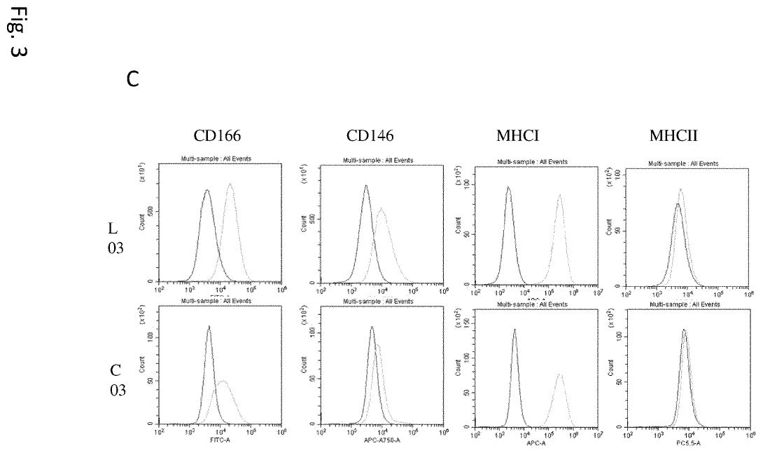

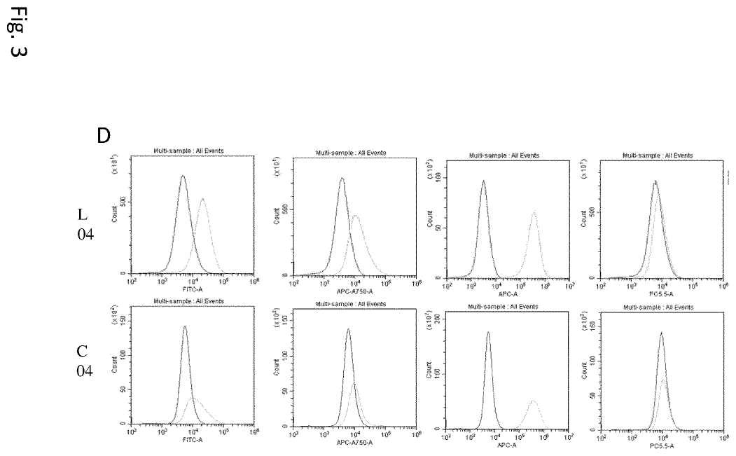

Results are reported in FIG. 3A-E. The black curve is indicative of the unstained cells. The grey curve is indicative of the expression of each marker in the cell population.

The data clearly show that CD166 positive cells are increased in the samples cultured according to the present invention (L) with respect to the control (C) (1.5-fold higher, Table 2). The same is true for the percentage of CD146 positive cells (5.7-fold higher, Table 3). The cells cultured according to the present invention express MHC-I (Table 4), but they are almost completely negative for MHC-II (Table 5), showing an immunogenicity profile resembling the one of mesenchymal stromal cells, considered poorly immunogenic.

TABLE 2
CD166

| | | % positive cells | Ratio |
|---|---|---|---|
| Donor01 | L | 94.5 | 1.3 |
| | C | 71.3 | |
| Donor02 | L | 69.7 | 0.9 |
| | C | 76.4 | |
| Donor03 | L | 60.4 | 1.3 |
| | C | 46.8 | |
| Donor04 | L | 43.3 | 1.1 |
| | C | 38.0 | |
| Donor05 | L | 83.6 | 3.0 |
| | C | 28.1 | |

TABLE 3
CD146

| | | % positive cells | Ratio |
|---|---|---|---|
| Donor05 | L | 72.2 | 2.3 |
| | C | 30.8 | |
| Donor06 | L | 47.5 | 5.4 |
| | C | 8.7 | |
| Donor14 | L | 41.7 | 5.6 |
| | C | 7.4 | |
| Donor16 | L | 38.5 | 5.2 |
| | C | 10.3 | |
| Donor17 | L | 44.2 | 9.8 |
| | C | 4.5 | |

TABLE 4
MHCI

| | | % Positive cells |
|---|---|---|
| Donor01 | L | 99.5 |
| | C | 99.9 |
| Donor02 | L | 99.9 |
| | C | 99.9 |
| Donor03 | L | 99.8 |
| | C | 99.9 |
| Donor04 | L | 99.8 |
| | C | 99.8 |
| Donor05 | L | 99.7 |
| | C | 99.9 |

TABLE 5
MHCII

| | | % Positive cells |
|---|---|---|
| Donor01 | L | 2.6 |
| | C | 12.7 |
| Donor02 | L | 2.4 |
| | C | 2.7 |
| Donor03 | L | 1.8 |
| | C | 1.7 |
| Donor04 | L | 2.4 |
| | C | 2.2 |
| Donor05 | L | 2.6 |
| | C | 1.7 |

### Example 4: Histological and Immunohistochemical Scores

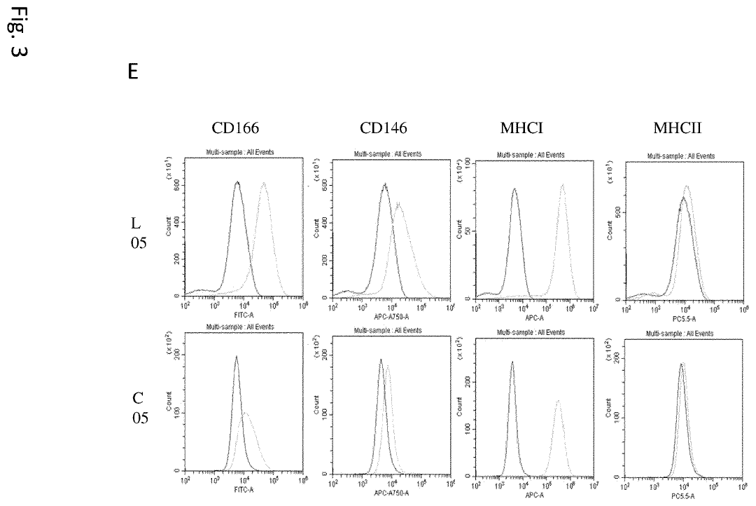

The cells cultured from each donor according to the present invention (L) are able to aggregate and generate pellets, while the cells cultured at standard density (C) are not always able to aggregate into stable pellets. After chondrogenic induction in pellet culture, the presence of a denser matrix in pellets from cells cultured according to (L) is observed compared to pellets from cells cultured at standard density (C), which often disintegrate when cut with a microtome. A typical chondrogenic round cell morphology, a higher matrix deposition (assessed as higher cell distance), and a higher amount of glycosaminoglycans (detected by alcian blue staining and indicative of matrix quality) are detected in pellets from cells cultured according to (L) in comparison with pellets from cells cultured at standard density (C). The scores related to these parameters are used to calculate the Bern score, which is higher in pellets obtained from cells cultured according to (L). Concerning the quality of the matrix, immunohistochemical analysis reveal a superior presence of type I and II collagens, that together with glycosaminoglycans are the fundamental constituents of the cartilaginous matrix, in pellets from cells cultured according to (L) in comparison with (C). Type II collagen appears more homogeneously distributed in cells cultured according to the invention (L). Table 6 shows the results of the histological and immunohistochemical analyses. Representative images showing the haematoxylin/eosin (HE), alcian blue staining, and type I and II collagen immunohistochemistry are shown in FIG. 4.

TABLE 6

| | | Cell | Cell | Alcian | Total Bern | Type I | Type II |
|---|---|---|---|---|---|---|---|
| | | distance | morphology | blue | score | collagen | collagen |
| Donor01 | L | 2.3 | 2.3 | 2.3 | 7.0 | 3.0 | 2.0 |
| | C | 0.3 | 1.0 | 1.5 | 2.8 | 1.0 | 1.0 |
| Donor02 | L | 2.2 | 2.7 | 2.2 | 7.0 | 2.0 | 2.8 |
| | C | 0.8 | 0.0 | 2.3 | 3.2 | 0.0 | 2.0 |
| Donor03 | L | 3.0 | 3.0 | 3.0 | 9.0 | 3.0 | 3.0 |
| | C | 0.0 | 0.0 | 0.0 | 0.0 | 0.0 | 0.0 |
| Donor04 | L | 2.3 | 2.3 | 2.2 | 6.8 | 2.0 | 2.3 |
| | C | 1.3 | 2.5 | 1.2 | 5.0 | 1.3 | 2.0 |
| Donor05 | L | 2.0 | 2.0 | 1.5 | 5.5 | 1.0 | 1.0 |
| | C | 0.5 | 0.0 | 0.0 | 0.5 | 1.0 | 1.0 |
| Mean ± SD | L | 2.4 ± 0.4 | 2.5 ± 0.4 | 2.2 ± 0.5 | 7.1 ± 1.3 | 2.2 ± 0.8 | 2.2 ± 0.8 |
| | C | 0.6 ± 0.5 | 0.7 ± 1.1 | 1.0 ± 1.0 | 2.3 ± 2.1 | 0.7 ± 0.6 | 1.2 ± 0.8 |
| t test L vs C | | ** | * | * | * | * | n.s. |

### Example 5: Expression of Specific Markers

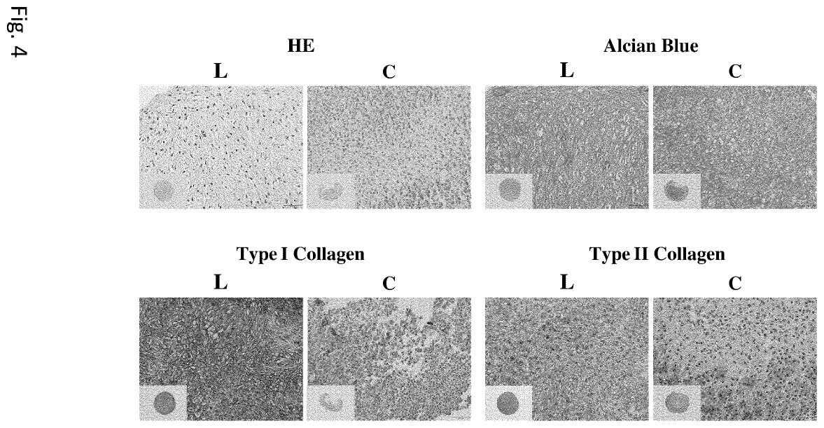

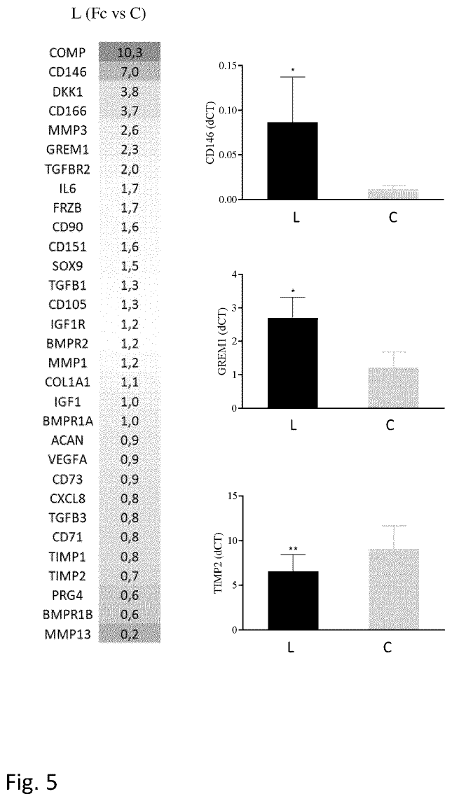

The expression of a panel of genes in the cell populations is showed in FIG. 5. A significantly higher expression of the surface marker CD146 and of GREM1, a marker involved in the downregulation of hypertrophy, is observed in the cells cultured according to the present invention (L) with respect to the control cells (C). On the contrary, TIMP2, a marker of cartilage remodelling, is significantly downregulated in the cells cultured according to the present invention (L) with respect to the control cells (C).

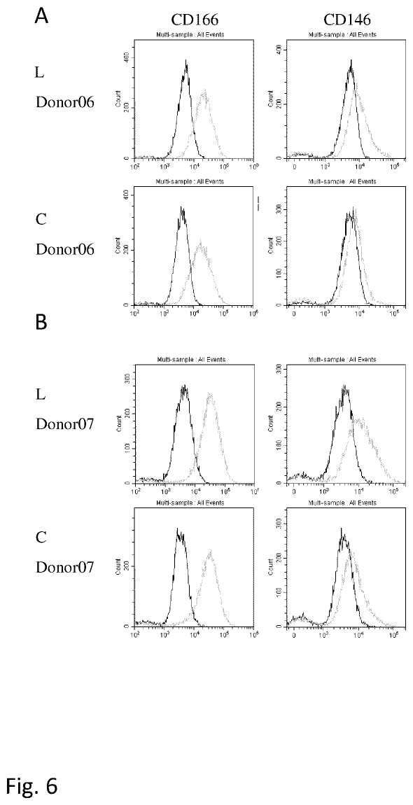

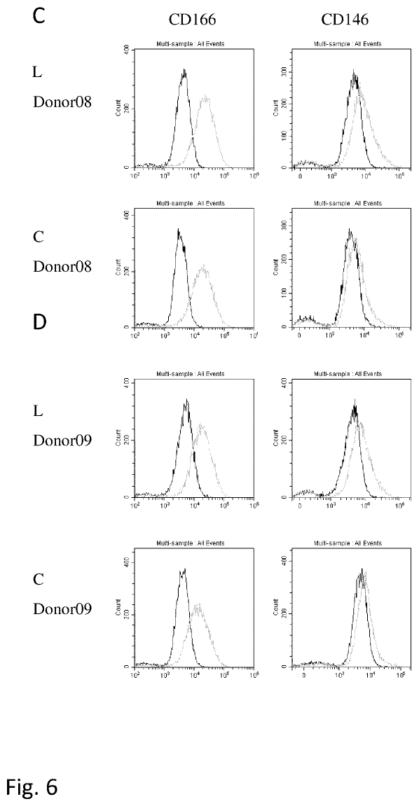

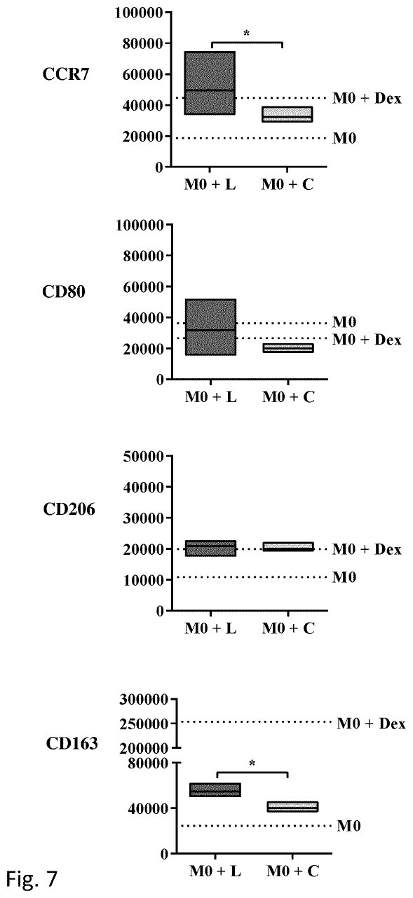

The surface expression of CD146 and CD166 in cells cultured according to the invention (L) or according to the control condition (C) for 14 days was evaluated. Results are reported in FIG. 6 A-D. The black curve is indicative of the unstained cells. The grey curve is indicative of the expression of each marker in the cell population. Table 7 and Table 8 show the results of the flow cytometry analysis in term of percentage of positive cells and MFI for each donor. The data show that CD146 and CD166 positive cells are increased in the samples cultured according to the present invention (L) with respect to the control (C).

TABLE 7
CD166

| CD166 | | % positive cells | Ratio (%) | MFI |
|---|---|---|---|---|
| Donor06 | L | 63.29 | 1.1 | 25192 |
| | C | 56.78 | | 22238 |
| Donor07 | L | 84.01 | 1.0 | 38812 |
| | C | 84.07 | | 36543 |
| Donor08 | L | 70.18 | 1.1 | 27813 |
| | C | 62.04 | | 24250 |
| Donor09 | L | 50.11 | 1.1 | 23000 |
| | C | 44.74 | | 17741 |
| Mean + SD | L | 66.90 ± 14.12 | | 28704 ± 7020 |
| | C | 63.62 ± 19.71 | | 26178 ± 9548 |
| t test L vs C | | n.s. | | * |

TABLE 8
CD146

| CD146 | | % positive cells | Ratio (%) | MFI |
|---|---|---|---|---|
| Donor06 | L | 18.48 | 4.5 | 11814 |
| | C | 4.15 | | 8486 |
| Donor07 | L | 36.25 | 3.9 | 16460 |
| | C | 9.36 | | 9046 |
| Donor08 | L | 19.96 | 3.6 | 10971 |
| | C | 4.71 | | 7272 |
| Donor09 | L | 16.05 | 3.8 | 9819 |
| | C | 4.20 | | 7903 |
| Mean + SD | L | 21.94 ± 9.60 | | 12266 ± 2913 |
| | C | 6.09 ± 2.84 | | 8074 ± 899 |
| t test L vs C | | * | | * |

### Example 6: Immunomodulatory Ability

Table 9 shows the MFI of the expression of macrophage surface markers after co-culture with chondrocytes. Interestingly, an increase of the CD163 and CCR7 expression on the macrophage surface has been observed, suggesting an anti-inflammatory and remodelling activities of these cells when co-cultured with cells cultured according to the present invention (L) with respect to the control cells (C). Of note, an increase of the same markers is observed on macrophages treated with dexamethasone, an anti-inflammatory drug typically used for the symptomatic treatment of the OA. Additionally, the cells cultured according to the present invention (L) upregulate the expression of the M2a marker CD206, similarly to the pharmacological treatment with dexamethasone.

TABLE 9

| | | CCR7 | CD80 | CD206 | CD163 |
|---|---|---|---|---|---|
| Donor05 | L | 48970 | 33299 | 21602 | 58803 |
| | C | 28657 | 17161 | 19542 | 37403 |
| Donor06 | L | 33635 | 15332 | 17494 | 52011 |
| | C | 28976 | 18494 | 19017 | 43579 |
| Donor14 | L | 38717 | 25351 | 20122 | 49829 |
| | C | 32367 | 23511 | 19946 | 46062 |
| Donor16 | L | 50716 | 32006 | 22077 | 50170 |
| | C | 31887 | 18554 | 19436 | 36445 |
| Donor17 | L | 74873 | 52087 | 22794 | 62401 |
| | C | 39277 | 22161 | 22199 | 36806 |
| Mean + SD | L | 49382 ± | 31615 ± | 20818 ± | 54643 ± |
| | | 15918 | 13476 | 2100 | 5648 |
| | C | 32233 ± | 19976 ± | 20028 ± | 40059 ± |
| | | 4276 | 2712 | 1258 | 4448 |
| t test L vs C | | * | n.s. | n.s. | * |

1. A method to obtain a cartilage cell population enriched in chondroprogenitor cells (CPCs), said method comprising:
Making available cartilage fragments;
Isolating, from said fragments, a population of cartilage cells;
Plating said cartilage cells at standard density in culture medium;
Culturing said cartilage cells;
Once confluence is reached, collecting the cells, and plating them in culture medium at low density, between 10 and 500 cells/cm², culturing until use.

2. The method according to claim 1, wherein said culture medium for plating at standard or at low density is independently supplemented with blood derivatives, selected from the group comprising platelet concentrates, platelets lysate (PL), serum.

3. The method according to claim 2, wherein said blood derivatives are of human origin.

4. The method according to claim 2, wherein said blood derivative is hPL.

5. The method according to claim 2, wherein said blood derivatives are added at a % comprised between 2% and 20% (V/V), or between 5% and 10% (V/V).

6. The method according to claim 1, wherein said standard density is comprised between 3.000 and 50.000 cells/cm², or between 5.000-30.000 cells/cm², or 6.000 and 10.000 cells/cm².

7. The method according to claim 1, wherein said low density is comprised between 20 and 100 cells/cm², or about 50 cells/cm².

8. A population of cartilage cells obtained according to the method of claim 1.

9. A population of cartilage cells enriched in CPCs characterized by an increased expression of the mesenchymal surface marker CD146.

10. A population of cartilage cells enriched in CPCs obtained by means of the methods of claim 1 for use in the treatment of chondral lesions or articular degeneration, among which OA and camptodactyly-arthropathy-coxa vara-pericarditis syndrome.

11. A population of cartilage cells enriched in CPCs for use according to claim 10, wherein said treatment is autologous.

12. A population of cartilage cells enriched in CPCs for use according to claim 10, wherein said treatment is allogeneic/heterologous.

13. A population of cartilage cells enriched in CPCs according to claim 9, for use in the treatment of chondral lesions or articular degeneration, among which OA and camptodactyly-arthropathy-coxa vara-pericarditis syndrome.

14. A population of cartilage cells enriched in CPCs for use according to claim 13, wherein said treatment is autologous.

15. A population of cartilage cells enriched in CPCs for use according to claim 13, wherein said treatment is allogeneic/heterologous.
